(19) United States
(12) Patent Application Publication
(10) Pub. No.: US 2025/0170294 A1
(43) Pub. Date: May 29, 2025

(54) APPARATUS FOR STERILIZATION AND DEODORIZATION AND REFRIGERATOR HAVING THE SAME

(71) Applicant: Samsung Electronics Co., Ltd., Suwon-si (KR)

(72) Inventors: Myungju SHIN, Suwon-si (KR); Sanghoon LEE, Suwon-si (KR); Donghyo KIM, Suwon-si (KR); Jinkook YOON, Suwon-si (KR); Bokyung LEE, Suwon-si (KR); Jisook LEE, Suwon-si (KR); Youngchul KO, Suwon-si (KR); Kisup LEE, Suwon-si (KR)

(21) Appl. No.: 18/930,383

(22) Filed: Oct. 29, 2024

Related U.S. Application Data
(63) Continuation of application No. PCT/KR2024/015291, filed on Oct. 8, 2024.

(30) Foreign Application Priority Data
Nov. 29, 2023 (KR) 10-2023-0170182

(51) Int. Cl. A61L 9/20; F25D 11/02

(52) U.S. Cl. CPC A61L 9/20; F25D 11/02; A61L 2209/12; A61L 2209/14; F25D 2323/0024

(57) ABSTRACT

A refrigerator comprises: a cabinet; a storage compartment disposed inside the cabinet; a cold air duct disposed behind the storage compartment; and a sterilization and deodorization apparatus mounted inside the storage compartment, and configured to draw air discharged from the cold air duct, sterilize and deodorize the air, and discharge the sterilized and deodorized air into the storage compartment. The sterilization and deodorization apparatus comprises: an inlet connected to a duct outlet of the cold air duct; a deodorizing filter configured to deodorize the air flowing into the inlet; a sterilizing lamp disposed between the inlet and the deodorizing filter and configured to sterilize the air flowing into the inlet; an outlet configured to discharge air sterilized and deodorized by the sterilizing lamp and the deodorizing filter into the storage compartment; and a deodorizing lamp disposed opposite to the sterilizing lamp with respect to the deodorizing filter and configured to activate the deodorizing filter.

### CROSS-REFERENCE TO RELATED APPLICATIONS

This application is a continuation of International Application No. PCT/KR2024/015291 designating the United States, filed on Oct. 8, 2024, in the Korean Intellectual Property Receiving Office and claiming priority to Korean Patent Application No. 10-2023-0170182, filed on Nov. 29, 2023, in the Korean Intellectual Property Office, the disclosures of each of which are incorporated by reference herein in their entireties.

### BACKGROUND

### Field

The disclosure relates to a refrigerator including a sterilization and deodorization apparatus in which a sterilizing lamp and a deodorizing filter are provided inside one housing.

### Description of Related Art

A refrigerator is a device that keeps food fresh by including a cabinet including a storage compartment provided to store food, and a cold air supply system configured to supply cold air to the storage compartment. The storage compartment includes a refrigerating compartment maintained at temperature of about 0 to 5° C. to keep food refrigerated, and a freezing compartment maintained at temperature of about 0 to −30° C. to keep food frozen. A door may be disposed on a front surface of the cabinet to open and close the storage compartment. The door may be rotatably provided on the front surface of the cabinet to open and close the storage compartment. The door may be a drawer type door to open and close the storage compartment.

Horticultural products such as vegetables and fruits or agricultural and livestock products may be stored inside the storage compartment. During the distribution process or the process of storing the products in the storage compartment, spoilage may occur, causing bacteria to proliferate or causing bad odors. Even if a source of the odor is removed, the odor may remain inside the storage compartment.

In order to remove bacteria or bad odors remaining inside the storage compartment, a sterilization and deodorization apparatus configured to sterilize and deodorize air may be installed inside the storage compartment.

### SUMMARY

Embodiments of the disclosure provide a refrigerator including a sterilization and deodorization apparatus in which a sterilizing lamp and a deodorizing filter are provided inside one housing.

Embodiments of the disclosure provide a refrigerator including a sterilization and deodorization apparatus capable of securing a sufficient sterilizing area and sterilizing time of air by placing a sterilizing lamp in a central portion of a housing.

Embodiments of the disclosure provide a refrigerator including a sterilization and deodorization apparatus capable of simultaneously ensuring sterilization and/or deodorization of air using a sterilizing lamp, by placing a sterilizing lamp between an inlet and a deodorizing filter.

Embodiments of the disclosure provide a refrigerator including a sterilization and deodorization apparatus capable of improving power consumption by sterilizing and/or deodorizing air using a sterilizing lamp.

Embodiments of the disclosure provide a refrigerator including a sterilization and deodorization apparatus capable of minimizing and/or reducing loss of air flow during sterilization and/or deodorization of air, by implementing a straight flow path in which air flowing into an inlet flows along the inlet, a sterilizing lamp, a deodorizing filter, and an outlet.

Embodiments of the disclosure provide a refrigerator including a sterilization and deodorization apparatus capable of minimizing and/or reducing a volume by allowing a distance between an inlet and a sterilizing lamp to be greater than a distance between the sterilizing lamp and a deodorizing filter.

Embodiments of the disclosure provide a refrigerator including a sterilization and deodorization apparatus capable of preventing and/or reducing ultraviolet rays, which are emitted from a sterilizing lamp, from leaking out of a housing through an inlet and an outlet, by installing a rib on the housing.

Embodiments of the disclosure provide a refrigerator including a sterilization and deodorization apparatus capable of preventing and/or reducing dew from forming on a wall of a housing adjacent to a storage compartment by attaching an insulating tape to the wall of the housing adjacent to the storage compartment.

Embodiments of the disclosure provide a refrigerator including a sterilization and deodorization apparatus capable of preventing and/or reducing dew from forming on a wall of a housing adjacent to a storage compartment by allowing at least a portion of a metal heat dissipation support, which supports a sterilizing lamp, to be in contact with the wall of the housing adjacent to the storage compartment.

Embodiments of the disclosure provide a refrigerator including a sterilization and deodorization apparatus capable of preventing and/or reducing loss of output from a sterilizing lamp by attaching an insulating tape to a wall of a housing adjacent to a storage compartment.

Embodiments of the disclosure provide a refrigerator including a sterilization and deodorization apparatus capable of increasing a lifespan of a sterilizing lamp by providing a heat dissipation support, which supports the sterilizing lamp, in order to dissipate heat generated from the sterilizing lamp.

Embodiments of the disclosure provide a refrigerator including a sterilization and deodorization apparatus capable of preventing and/or reducing photodeterioration of a housing by attaching an insulating tape to a wall of the housing adjacent to a storage compartment.

According to an example embodiment, the present disclosure provides a refrigerator including: a cabinet, a storage compartment disposed inside the cabinet, a cold air duct disposed behind the storage compartment, and a sterilization and deodorization apparatus mounted inside the storage compartment, and configured to draw air discharged from the cold air duct, and sterilize and deodorize the air, and discharge the sterilized and deodorized air into the storage compartment, wherein sterilization and deodorization apparatus includes: an inlet connected to a duct outlet of the cold air duct, a deodorizing filter configured to deodorize the air flowing into the inlet, a sterilizing lamp disposed between the inlet and the deodorizing filter and configured to sterilize the air flowing into the inlet, an outlet configured to discharge air sterilized and deodorized by the sterilizing lamp and the deodorizing filter into the storage compartment, and a deodorizing lamp disposed opposite to the sterilizing lamp with respect to the deodorizing filter and configured to activate the deodorizing filter.

According to an example embodiment, the present disclosure provides a sterilization and deodorization apparatus configured to draw air, discharged from a duct outlet of a cold air duct disposed behind a storage compartment, and sterilize and deodorize the air, and discharge the sterilized and deodorized air into the storage compartment, the sterilization and deodorization apparatus including: a housing mounted inside the storage compartment, an inlet connected to the duct outlet, a deodorizing filter configured to deodorize the air flowing into the inlet, a sterilizing lamp having a cylindrical shape and disposed between the inlet and the deodorizing filter and configured to sterilize the air flowing into the inlet, an outlet configured to discharge air sterilized and deodorized by the sterilizing lamp and the deodorizing filter into the storage compartment, and a deodorizing lamp disposed opposite to the sterilizing lamp with respect to the deodorizing filter and configured to activate the deodorizing filter.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a perspective view illustrating a refrigerator with an open door according to various embodiments;

FIG. 2 is a diagram illustrating a side cross-sectional view of the refrigerator according to various embodiments;

FIG. 3 is a cross-sectional view illustrating a state in which a sterilization and deodorization apparatus mounted on an upper wall of a refrigerating compartment is connected to a cold air duct according to various embodiments;

FIG. 4 is a perspective view illustrating the sterilization and deodorization apparatus mounted on the upper wall of the refrigerating compartment according to various embodiments;

FIG. 5 is a perspective view illustrating the sterilization and deodorization apparatus disassembled from the upper wall of the refrigerating compartment according to various embodiments;

FIG. 6 is a perspective view illustrating the sterilization and deodorization apparatus according to various embodiments;

FIG. 7 is an exploded perspective view of the sterilization and deodorization apparatus according to various embodiments;

FIG. 8 is a diagram illustrating a top plan view of an inside of a second housing in which a sterilizing lamp, a deodorizing filter, and a deodorizing lamp are installed and an insulating tape is attached according to various embodiments;

FIG. 9 is a cross-sectional view illustrating a blocking rib provided in a portion adjacent to an inlet of the sterilization and deodorization apparatus according to various embodiments;

FIG. 10 is a cross-sectional view illustrating a state in which a first blocking rib provided in a portion adjacent to the inlet of the sterilization and deodorization apparatus is formed in front of a second blocking rib according to various embodiments;

FIG. 11 is a cross-sectional view illustrating a state in which a second blocking rib provided in a portion adjacent to the inlet of the sterilization and deodorization apparatus is formed in front of a first blocking rib according to various embodiments;

FIG. 12 is an exploded perspective view of the sterilization and deodorization apparatus including a heat dissipation support supporting the sterilizing lamp according to various embodiments;

FIG. 13 is a perspective view illustrating a state in which the heat dissipation support supports the sterilizing lamp according to various embodiments;

FIG. 14 is a cross-sectional view of the sterilization and deodorization apparatus including the heat dissipation support supporting the sterilizing lamp according to various embodiments;

FIG. 15 is a diagram illustrating a top plan view of the inside of the second housing in which the sterilizing lamp, the deodorizing filter, and the deodorizing lamp are installed, the heat dissipation support supports the sterilizing lamp, and the insulating tape is attached, according to various embodiments;

FIG. 16 is a perspective view illustrating a state in which the heat dissipation support supporting the sterilizing lamp includes an extension portion extending from a support portion according to various embodiments;

FIG. 17 is a cross-sectional view of the sterilization and deodorization apparatus including the heat dissipation support including the extension portion according to various embodiments;

FIG. 18 is a diagram illustrating a top plan view of the inside of the second housing in which the sterilizing lamp, the deodorizing filter, and the deodorizing lamp are installed, the heat dissipation support including the extension portion supports the sterilizing lamp, and the insulating tape is attached, according to various embodiments;

FIG. 19 is a perspective view illustrating a state in which the extension portion of the heat dissipation support includes an expansion portion having a width greater than a width of the support portion according to various embodiments;

FIG. 20 is a cross-sectional view of the sterilization and deodorization apparatus including the heat dissipation support including the expansion portion according to various embodiments;

FIG. 21 is a diagram illustrating a top plan view of the inside of the second housing in which the sterilizing lamp, the deodorizing filter, and the deodorizing lamp are installed, the heat dissipation support including the expansion portion supports the sterilizing lamp, and the insulating tape is attached, according to various embodiments;

FIG. 22 is a perspective view illustrating a state in which expansion portions of the heat dissipation support are connected to each other in a direction perpendicular to a flow direction of air according to various embodiments; and

FIG. 23 is a diagram illustrating a top plan view of the inside of the second housing in which the sterilizing lamp, the deodorizing filter, and the deodorizing lamp are installed, the heat dissipation support including the expansion portions, which are connected to each other in the direction perpendicular to the flow direction of air, supports the sterilizing lamp, and the insulating tape is attached, according to various embodiments.

### DETAILED DESCRIPTION

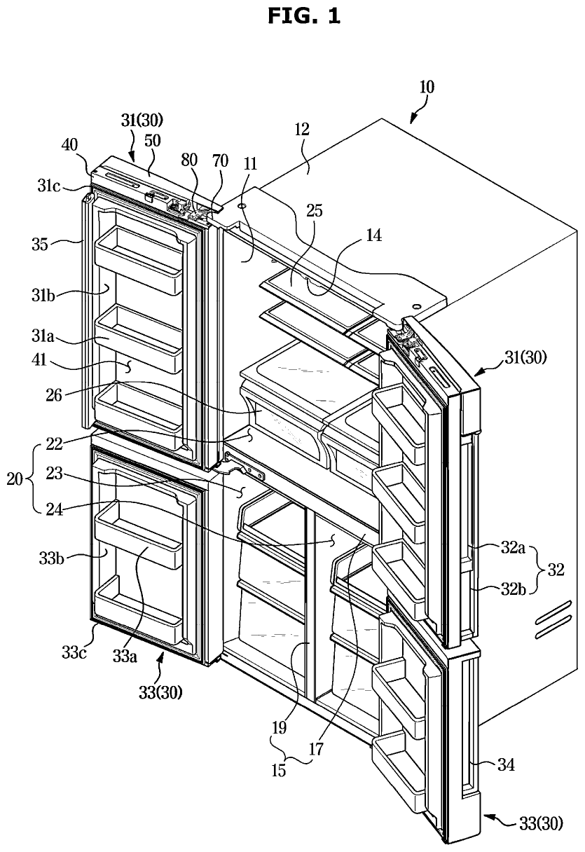

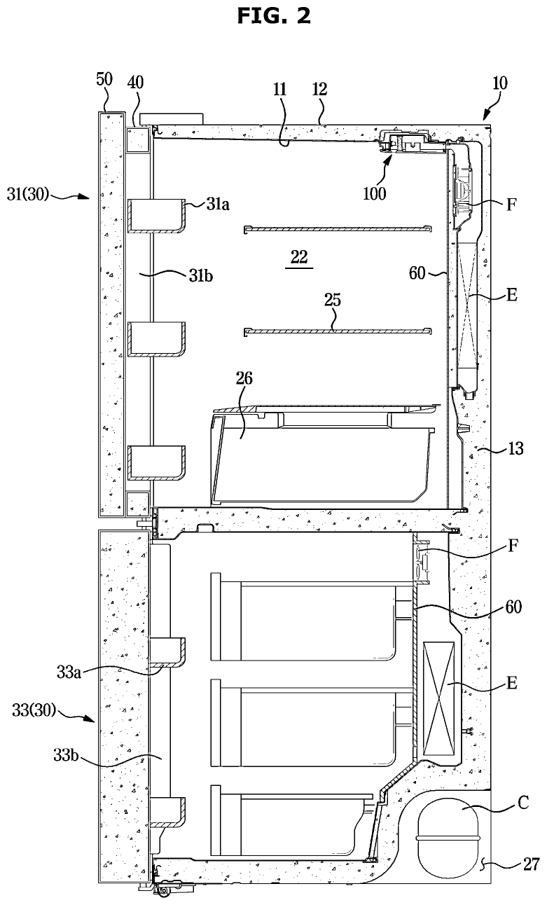

FIG. 1 is a perspective view illustrating a refrigerator with an open door according to various embodiments. FIG. 2 is a side cross-sectional view of the refrigerator according to various embodiments.

As illustrated in FIGS. 1 and 2, a refrigerator may include a cabinet 10, a storage compartment 20 vertically partitioned inside the cabinet 10, a door 30 configured to open and close the storage compartment 20, and a cold air supply device (not shown) configured to supply cold air to the storage compartment 20.

The cabinet 10 may include an inner case 11 forming the storage compartment 20, an outer case 12 coupled to an outside of the inner case 11 to form an external appearance, and an insulation material 13 foamed between the inner case 11 and the outer case 12 to thermally insulate the storage compartment 20.

A machine room 27, in which a compressor C configured to compress a refrigerant and a condenser (not shown) configured to condense the refrigerant compressed by the compressor C are installed, may be provided at a rear lower portion of the cabinet 10.

The cold air supply device may include the compressor C installed in the machine room 27 to compress the refrigerant, the condenser (not shown) installed in the machine room 27 to condense the refrigerant, an expansion valve (not shown) configured to expand the refrigerant, which is expanded by the condenser, an evaporator E installed at the rear of the storage compartment 20 to generate cold air, a fan F provided to allow the cold air generated in the evaporator E to be discharged into the storage compartment 20, and a cold air duct 60 installed at the rear of the storage compartment 20 to discharge the cold air, which is guided by the fan F, to the storage compartment 20.

The storage compartment 20 may be divided into a plurality of spaces by a partition 15, and a plurality of shelves 25 and storage containers 26 may be provided in the storage compartment 20 to store food and the like.

The storage compartment 20 may be divided into a plurality of storage compartments 22, 23, and 24 by the partition 15. The partition 15 may include a first partition 17 horizontally coupled to the inside of the storage compartment 20 to divide the storage compartment 20 into an upper storage compartment 22 and lower storage compartments 23 and 24, and a second partition 19 vertically coupled to the inside of the lower storage compartments 23 and 24 to divide the lower storage compartments 23 and 24 into a first storage compartment 23 and a second storage compartment 24.

The partition 15 including a T-shape formed by the first partition 17 and the second partition 19 coupled to each other may divide the storage compartment 20 into three spaces. Among the upper storage compartment 22 and the lower storage compartments 23 and 24 divided by the first partition 17, the upper storage compartment 22 may be used as a refrigerating compartment, and the lower storage compartments 23 and 24 may be used as a freezing compartment.

All the lower storage compartments 23 and 24 may be used as the freezing compartment. Alternatively, the first storage compartment 23 may be used as the freezing compartment and the second storage compartment 24 may be used as the refrigerating compartment. Alternatively, the first storage compartment 23 may be used as the freezing compartment, and the second storage compartment 24 may be used as the freezing compartment and the refrigerating compartment.

The division of the storage compartment 20 as described above is an example, and each storage compartment 22, 23, and 24 may be used differently from the above configuration.

The refrigerating compartment 22 and the freezing compartments 23 and 24 may be opened and closed by the door 30 rotatably coupled to the cabinet 10, respectively.

The door 30 may include a pair of refrigerating compartment doors 31 rotatably coupled to the cabinet 10 to open and close the refrigerating compartment 22, and a pair of freezing compartment doors 33 rotatably coupled to the cabinet 10 to open and close the freezing compartments 23 and 24.

The pair of refrigerating compartment doors 31 may be respectively opened and closed through a pair of refrigerating compartment door handles 32 including a first door handle 32a or a second door handle 32b. The refrigerating compartment 22 may be opened and closed by the pair of refrigerating compartment doors 31, and a rotating rod 35 may be installed on at least one of the pair of refrigerating compartment doors 31 so as to seal between the pair of refrigerating compartment doors 31 without a gap therebetween which may occur in response to closing the refrigerating compartment doors 31. The rotating rod 35 may be rotatably coupled to at least one of the pair of refrigerating compartment doors 31. The rotating rod 35 may be guided by a rotation guide 14 formed in the cabinet 10, so as to rotate according to the opening and closing of the refrigerating compartment door 31.

The pair of freezing compartment doors 33 may be respectively opened and closed by a freezing compartment door handle 34. A sliding door may be applied to the door configured to open and close the freezing compartments 23 and 24.

Door shelves 31a and 33a in which food is stored may be provided on rear surfaces of the pair of refrigerating compartment doors 31 and the pair of freezing compartment doors 33, respectively.

The door shelves 31a and 33a may respectively include shelf supports 31b and 33b extending vertically from the doors 31 and 33 to support the door shelves 31a and 33a on left and right sides of the door shelves 31a and 33a. The shelf supports 31b and 33b may extend from the doors 31 and 33, respectively. The shelf supports 31b and 33b may be removably provided on the doors 31 and 33, respectively, as a separate configuration.

In addition, first gaskets 31c and 33c may be respectively provided on edges of the rear surface of the doors 31 and 33 to seal a gap with the cabinet 10 in response to closing of the doors 31 and 33. The first gaskets 31c and 33c may be installed in a loop shape along the edges on the rear surface of each of the doors 31 and 33, and a magnet (not shown) may be included in the first gaskets 31c and 33c.

The refrigerating compartment door 31 may be provided as a double door including a first door 40 and a second door 50.

The first door 40 may be rotatably connected to the cabinet 10 by a first hinge 70 so as to open and close the refrigerating compartment 22. The above-described door shelf 31a,shelf support 31b, and first gasket 31c may be provided on the first door 40.

The first door 40 may include an opening 41 that is formed to allow a user to approach the door shelf 31a to insert or withdraw food while the first door 40 is closed. The opening 41 may be formed to penetrate the first door 40 and may be opened and closed by the second door 50.

The second door 50 may be disposed in front of the first door 40 to open and close the opening 41 of the first door 40, and the second door 50 may be rotatable in the same direction as the first door 40. It is illustrated that the second door 50 is rotatably supported by a second hinge 80 installed on the first door 40 and is rotatable with respect to the first door 40, but is not limited thereto. The second door 50 may be rotatable with respect to the cabinet 10 as the second hinge 80 is installed on the cabinet 10.

The second door 50 may include a second gasket (not shown) for maintaining airtightness with the first door 40. The second gasket may be installed in a loop shape along an edge of a rear surface of the second door 50, and a magnet (not shown) may be included therein.

In the refrigerating compartment 22. a sterilization and deodorization apparatus 100 configured to sterilize and deodorize air may be mounted on an upper wall of the refrigerating compartment 22. A detailed description of the sterilization and deodorization apparatus 100 will be provided below.

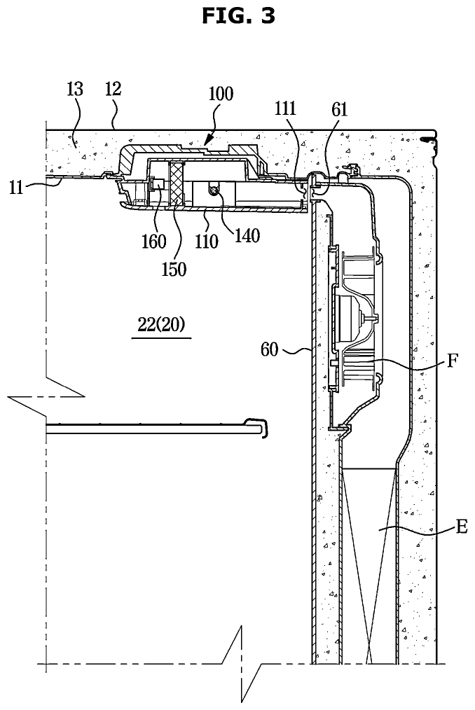

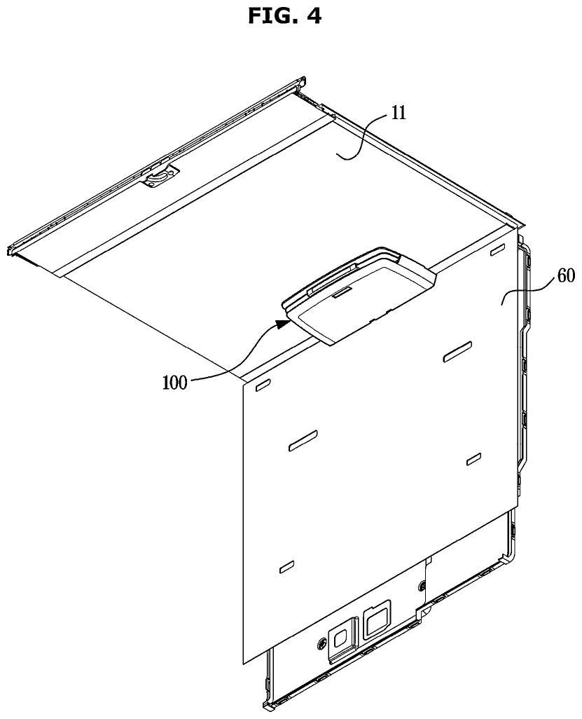

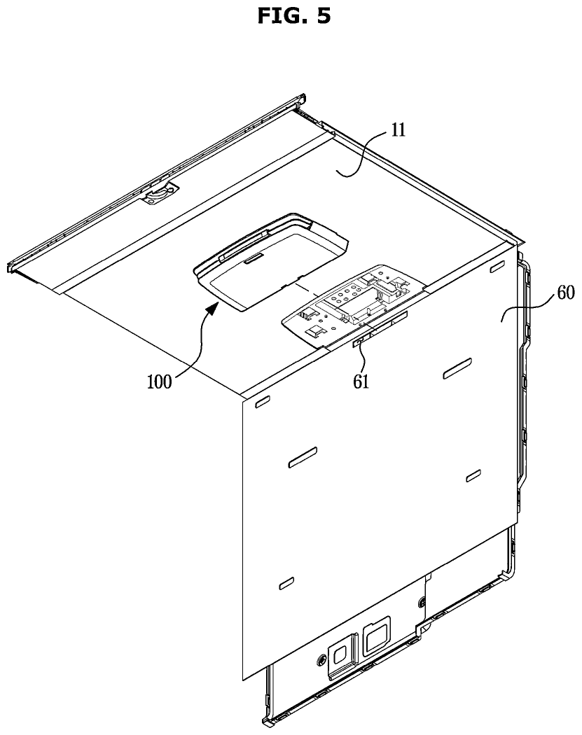

FIG. 3 is a cross-sectional view illustrating a state in which a sterilization and deodorization apparatus mounted on an upper wall of a refrigerating compartment is connected to a cold air duct according to various embodiments. FIG. 4 is a perspective view illustrating the sterilization and deodorization apparatus mounted on the upper wall of the refrigerating compartment according to various embodiments. FIG. 5 is a perspective view illustrating the sterilization and deodorization apparatus disassembled from the upper wall of the refrigerating compartment according to various embodiments.

As illustrated in FIGS. 3, 4 and 5 (which may be referred to as FIGS. 3 to 5), the sterilization and deodorization apparatus 100 may be mounted on the upper wall of the refrigerating compartment 22. The sterilization and deodorization apparatus 100 may include a housing 110 mounted on the upper wall of the refrigerating compartment 22. A detailed description of the housing 110 will be provided below.

The sterilization and deodorization apparatus 100 mounted on the upper wall of the refrigerating compartment 22 may be connected to the cold air duct 60. The cold air duct 60 may be disposed at the rear of the refrigerating compartment 22. Cold air generated in the evaporator E may be guided to move along the cold air duct 60 by the fan F. Cold air moving along the cold air duct 60 by the fan F may be discharged from the cold air duct 60 through a cold air outlet 61. Cold air discharged from the cold air outlet 61 may flow into the sterilization and deodorization apparatus 100. The cold air outlet 61 may be connected to an inlet 111 of the sterilization and deodorization apparatus 100. Accordingly, the cold air discharged through the cold air outlet 61 may flow into the sterilization and deodorization apparatus 100 through the inlet 111. Hereinafter cold air that is discharged from the cold air outlet 61 and flows into the inlet 111 will be referred to as air.

Air flowing into the sterilization and deodorization apparatus 100 through the inlet 111 may be sterilized by a sterilizing lamp 140 mounted inside the sterilization and deodorization apparatus 100. Air flowing into the sterilization and deodorization apparatus 100 through the inlet 111 may be deodorized by a deodorizing filter 150 mounted inside the sterilization and deodorization apparatus 100. Air that flows into the sterilization and deodorization apparatus 100 and is sterilized and deodorized may be discharged into the refrigerating compartment 22 through an outlet 170. A detailed configuration of the sterilization and deodorization apparatus 100 will be described below.

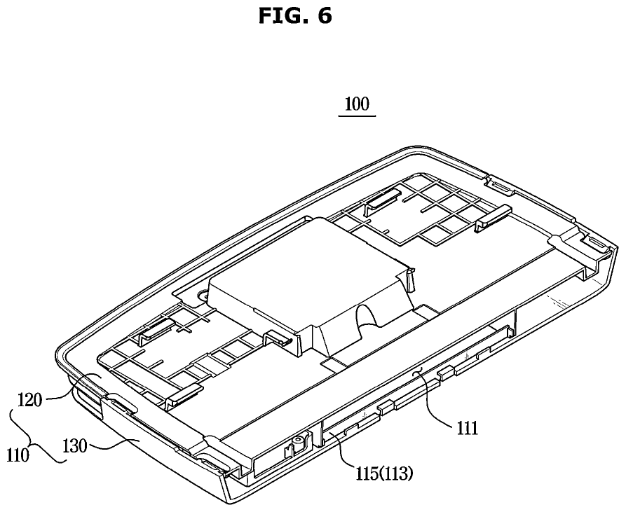

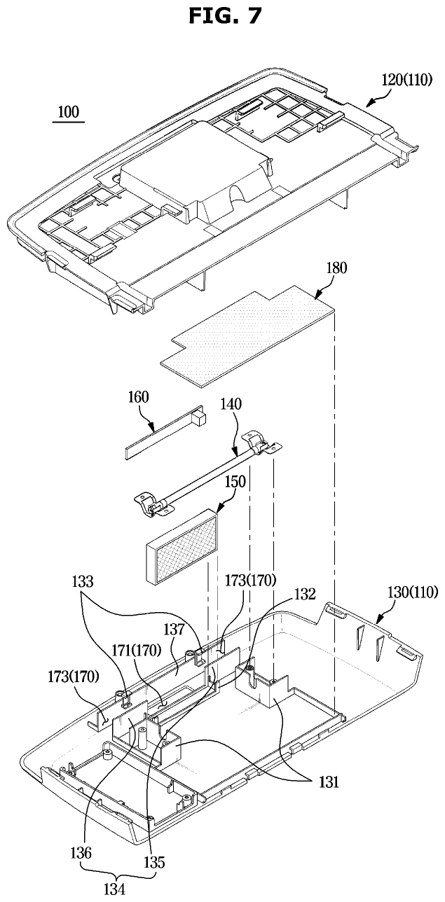

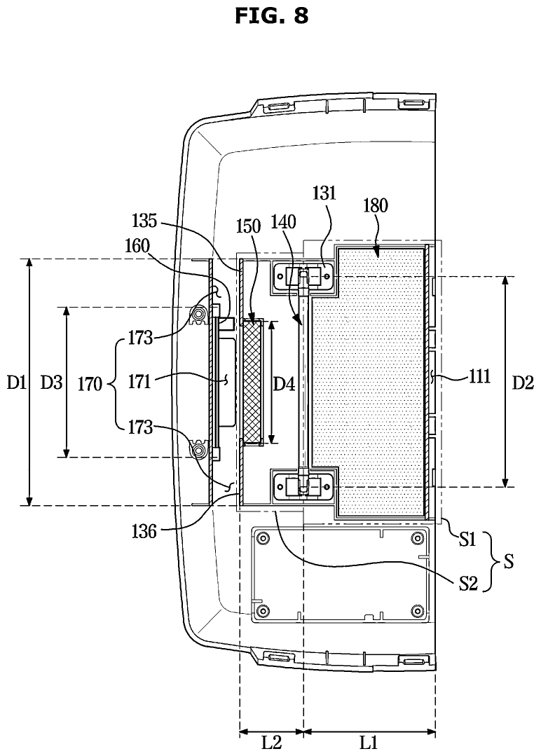

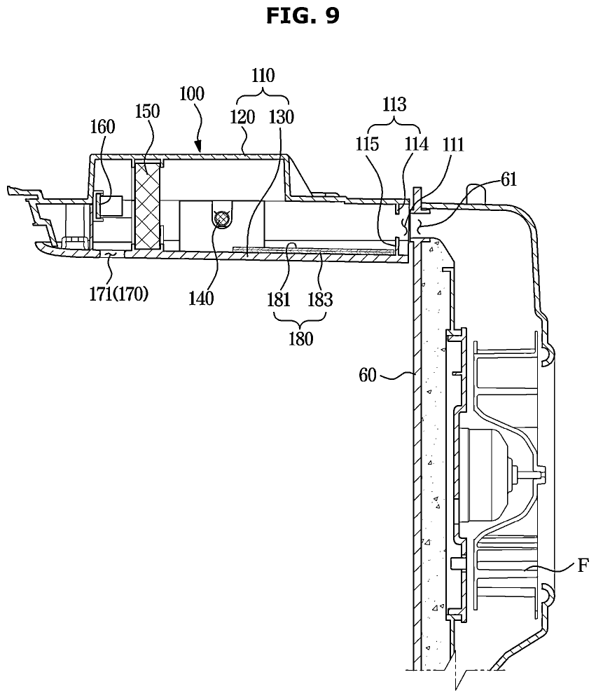

FIG. 6 is a perspective view illustrating the sterilization and deodorization apparatus according to various embodiments. FIG. 7 is an exploded perspective view of the sterilization and deodorization apparatus according to various embodiments. FIG. 8 is a diagram illustrating a top plan view of an inside of a second housing in which a sterilizing lamp, a deodorizing filter, and a deodorizing lamp are installed and an insulating tape is attached according to various embodiments. FIG. 9 is a cross-sectional view illustrating a blocking rib provided in a portion adjacent to an inlet of the sterilization and deodorization apparatus according to various embodiments.

Hereinafter when describing the sterilization and deodorization apparatus 100, a side in which the outlet 170 is disposed may represent a front side and a side in which the inlet 111 is disposed may represent a rear side based on the inlet 111 and the outlet 170. In addition, the sterilization and deodorization apparatus 100 is described as being mounted on the upper wall of the refrigerating compartment 22, but is not limited thereto. That is, the sterilization and deodorization apparatus 100 may be mounted on a part other than the upper wall of the refrigerating compartment 22. Alternatively, the sterilization and deodorization apparatus 100 may be installed inside the freezing compartments 23 and 24 rather than the refrigerating compartment 22. Alternatively, the sterilization and deodorization apparatus 100 may be installed inside both the refrigerating compartment 22 and the freezing compartments 23 and 24 (refer to FIG. 2)

As illustrated in FIGS. 6, 7, 8 and 9 (which may be referred to as FIGS. 6 to 9), the sterilization and deodorization apparatus 100 may include the housing 110. The housing 110 may be mounted on the upper wall of the refrigerating compartment 22. The housing 110 may include a first housing 120 mounted on the upper wall of the refrigerating compartment 22. The housing 110 may include a second housing 130 mounted on a lower portion of the first housing 120. The housing 110 may include the inlet 111 through which air flows. The inlet 111 may be formed by the first housing 120 and the second housing 130. The inlet 111 may be connected to the duct outlet 61 of the cold air duct 60. Air discharged from the duct outlet 61 of the cold air duct 60 may flow into the inlet 111. Air flowing into the housing 110 through the inlet 111 may be sterilized and deodorized by the sterilizing lamp 140 and the deodorizing filter 150 mounted inside the housing 110. The sterilized and deodorized air may be discharged into the refrigerating compartment 22 through the outlet 170 (refer to FIGS. 2 and 3).

The housing 110 may include a blocking rib 113 provided to prevent and/or reduce ultraviolet rays emitted from the sterilizing lamp 140 from leaking out of the housing 110 through the inlet 111. The air flowing into the housing 110 may be sterilized by the ultraviolet rays emitted from the sterilizing lamp 140 mounted inside the housing 110. The ultraviolet rays emitted from the sterilizing lamp 140 may be prevented and/or reduced from leaking into the cold air duct 60 through the inlet 111 by the blocking rib 113.

The blocking rib 113 may be provided in the first housing 120 and the second housing 130 adjacent to the inlet 111, respectively. The blocking rib 113 may include a first blocking rib 114 formed in the first housing 120. The first blocking rib 114 may be formed to extend downward from an upper wall of the first housing 120.

The blocking rib 113 may include a second blocking rib 115 formed in the second housing 130. The second blocking rib 115 may be formed to extend upward from a lower wall of the second housing 130.

The first blocking rib 114 and the second blocking rib 115 may be formed at the same position with respect to a flow direction of air flowing into the inlet 111. Accordingly, a space may be formed between the first blocking rib 114 and the second blocking rib 115 to allow the air flowing into the inlet 111 to flow into the housing 110. That is, the first blocking rib 114 and the second blocking rib 115 may be spaced apart from each other.

Some of the ultraviolet rays emitted from the sterilizing lamp 140 may be prevented and/or reduced from leaking into the inlet 111 by the blocking rib 113, but because the space is formed between the first blocking rib 114 and the second blocking rib 115, some of the ultraviolet rays may leak into the inlet 111. Ultraviolet rays flowing out of the inlet 111 through the space between the first blocking rib 114 and the second blocking rib 115 may flow into the cold air duct 60 through the duct outlet 61. A portion of the cold air duct 60 adjacent to the duct outlet 61 may be sterilized by the ultraviolet rays moving into the cold air duct 60 through the duct outlet 61.

The second housing 130 may include a sterilizing lamp mounting portion 131 on which the sterilizing lamp 140 is mounted. The sterilizing lamp mounting portion 131 may be provided on the lower wall of the second housing 130. The sterilizing lamp mounting portion 131 may be provided in a central portion of the lower wall of the second housing 130. The central portion of the lower wall of the second housing 130 may be a position with respect to the flow direction of air flowing into the housing 110. Both ends of the sterilizing lamp 140 having a cylindrical shape may be mounted on the sterilizing lamp mounting portion 131.

The second housing 130 may include a deodorizing filter mounting portion 132 on which the deodorizing filter 150 is mounted. The deodorizing filter mounting portion 132 may be provided on the lower wall of the second housing 130. The deodorizing filter mounting portion 132 may be provided in front of the sterilizing lamp mounting portion 131.

The second housing 130 may include a deodorizing lamp mounting portion 133 on which a deodorizing lamp 160 is mounted. The deodorizing lamp mounting portion 133 may be provided on the lower wall of the second housing 130. The deodorizing lamp mounting portion 133 may be provided in front of the deodorizing filter mounting portion 132. That is, the deodorizing lamp mounting portion 133 may be provided on an opposite side of the sterilizing lamp mounting portion 131 with respect to the deodorizing filter mounting portion 132.

Therefore, the inlet 111, the sterilizing filter mounting portion 131, the deodorizing filter mounting portion 132, and the deodorizing lamp mounting portion 133, which are provided in the housing 110, may be sequentially disposed according to the flow direction of air flowing into the housing 110. In other words, the inlet 111, the sterilizing filter 140, the deodorizing filter 150, and the deodorizing lamp 160 may be sequentially disposed along the flow direction of the air flowing into the housing 110.

The second housing 130 may include a deodorizing filter rib 134 disposed on the left and right sides of the deodorizing filter 150, respectively. That is, the deodorizing filter rib 134 may be provided on the left and right sides of the deodorizing filter mounting portion 132, respectively. The deodorizing filter rib 134 may prevent and/or reduce ultraviolet rays, which are emitted from the sterilizing lamp 140, from leaking to the outside through the outlet 170. In other words, the deodorizing filter rib 134 may prevent and/or reduce the ultraviolet rays emitted from the sterilizing lamp 140 from leaking into the refrigerating compartment 22 (refer to FIG. 2) through the outlet 170.

The deodorizing filter rib 134 may include a first deodorizing filter rib 135 disposed on the left side of the deodorizing filter 150. That is, the first deodorizing filter rib 135 may be disposed on the left side of the deodorizing filter mounting portion 132. At this time, the left side of the deodorizing filter 150 may be defined when viewed from the front of the housing 110 to the housing 110.

The deodorizing filter rib 134 may include a second deodorizing filter rib 136 disposed on the right side of the deodorizing filter 150. That is, the second deodorizing filter rib 136 may be disposed on the right side of the deodorizing filter mounting portion 132.

A length D1 between a left end of the first deodorizing filter rib 135 and a right end of the second deodorizing filter rib 136 may be greater than a length D2 of the sterilizing lamp 140. Because the length D1 between the left end of the first deodorizing filter rib 135 and the right end of the second deodorizing filter rib 136 is greater than the length D2 of the sterilizing lamp 140, it is possible to prevent and/or reduce ultraviolet rays emitted from the sterilizing lamp 140 from leaking into the refrigerating compartment 22 (refer to FIG. 2) through the outlet 170.

The second housing 130 may include a deodorizing lamp rib 137 disposed in front of the deodorizing lamp 160. That is, the deodorizing lamp rib 137 may be disposed in front of the deodorizing lamp mounting portion 133. The deodorizing lamp rib 137 may prevent and/or reduce ultraviolet rays emitted from the sterilizing lamp 140 from leaking to the outside through the outlet 170. That is, the deodorizing lamp rib 137 may prevent and/or reduce the ultraviolet rays emitted from the sterilizing lamp 140 from leaking into the refrigerating compartment 22 (refer to FIG. 2) through the outlet 170. Additionally, the deodorizing lamp rib 137 may prevent and/or reduce ultraviolet rays emitted from the deodorizing lamp 160 from leaking into the refrigerating compartment 22 (refer to FIG. 2) through the outlet 170. A length D3 of the deodorizing lamp rib 137 may be greater than a length D4 of the deodorizing filter 150.

The deodorizing filter rib 134 and the deodorizing lamp rib 137 may guide air, which flows into the housing 110 and is sterilized and deodorized, to be smoothly discharged through the outlet 170. That is, the air, which flows into the housing 110 and is sterilized and deodorized, may be guided through a space between the deodorizing filter rib 134 and the deodorizing lamp rib 137 and smoothly discharged through the outlet 170.

The sterilization and deodorization apparatus 100 may include the sterilizing lamp 140. The sterilizing lamp 140 may be an Ultraviolet-C (UVC) lamp configured emit ultraviolet rays. The sterilizing lamp 140 may be provided to have a cylindrical shape. Because the sterilizing lamp 140 has a cylindrical shape, ultraviolet rays emitted from the sterilizing lamp 140 may be emitted at 360 degrees. The sterilizing lamp 140 may be mounted on the sterilizing lamp mounting portion 131 provided in the second housing 130. Both ends of the sterilizing lamp 140 may be mounted on the sterilizing lamp mounting portion 131. The sterilizing lamp 140 may sterilize the air flowing into the inlet 111 by emitting ultraviolet rays. Air sterilized by the ultraviolet rays emitted from the sterilizing lamp 140 may be discharged into the refrigerating compartment 22 (refer to FIG. 2) through the outlet 170.

The sterilizing lamp 140 may be disposed between the inlet 111 and the deodorizing filter 150. Because the sterilizing lamp 140 is disposed between the inlet 111 and the deodorizing filter 150, a sterilizing area S, in which the air flowing into the inlet 111 is sterilized by ultraviolet rays emitted from the sterilizing lamp 140, may be formed in a section between the inlet 111 and the deodorizing filter 150. That is, the air flowing into the inlet 111 may be sterilized while flowing through the sterilization area S formed in the section between the inlet 111 and the deodorizing filter 150 and then discharged through the outlet 170. As described above, because the sterilizing lamp 140 configured to emit ultraviolet rays at 360-degrees is disposed in the central portion of the housing 110, the sterilizing area S for sterilizing the air may be secured as much as possible. In addition, because the sterilizing lamp 140 configured to emit ultraviolet rays at 360-degrees is disposed in the central portion of the housing 110, a sterilization time for sterilizing air may be secured as much as possible.

The sterilizing area S may include a first sterilizing area S1 formed in a section between the inlet 111 and the sterilizing lamp 140. The sterilizing area S may include a second sterilizing area S2 formed in a section between the sterilizing lamp 140 and the deodorizing filter 150.

A distance L1 between the inlet 111 and the sterilizing lamp 140 that is the first sterilizing area S1 may be greater than a distance L2 between the sterilizing lamp 140 and the deodorizing filter 150 that is the second sterilizing area S2. When the distance L1 between the inlet 111 and the sterilizing lamp 140 that is the first sterilizing area S1 is greater than the distance L2 between the sterilizing lamp 140 and the deodorizing filter 150 that is the second sterilizing area S2, it is possible to maximize and/or increase the sterilizing area S and to allow the sterilizing lamp 140 and the deodorizing filter 150 to be positioned in a certain distance. When the sterilizing lamp 140 and the deodorizing filter 150 are positioned within the certain distance, the deodorizing filter 150 may be activated by ultraviolet rays emitted from the sterilizing lamp 140. When the deodorizing filter 150 is activated by ultraviolet rays emitted from the sterilizing lamp 140, the air flowing into the inlet 111 may be deodorized by the deodorizing filter 150 and then discharged to the inside of the refrigerating compartment 22 (refer to FIG. 2) through the outlet 170. As described above, because air is sterilized and/or deodorized by a single lamp corresponding to the sterilizing lamp 140, the efficiency of power consumption may be increased. Further, an overall volume of the sterilization and deodorization apparatus 100 may be minimized and/or reduced.

The sterilization and deodorization apparatus 100 may include the deodorizing filter 150. The deodorizing filter 150 may be mounted on the deodorizing filter mounting portion 132 provided in the second housing 130. The deodorizing filter 150 may be disposed in front of the sterilizing lamp 140. That is, the deodorizing filter 150 may be disposed to allow the sterilizing lamp 140 to be positioned between the inlet 111 and the deodorizing filter 150. The deodorizing filter 150 may be activated by ultraviolet rays emitted from the sterilizing lamp 140. The deodorizing filter 150 activated by the ultraviolet rays emitted from the sterilizing lamp 140 may deodorize the air flowing into the inlet 111. The air deodorized by the deodorizing filter 150 may be discharged into the refrigerating compartment 22 (refer to FIG. 2) through the outlet 170.

The sterilization and deodorization apparatus 100 may include the deodorizing lamp 160. The deodorizing lamp 160 may be mounted on the deodorizing lamp mounting portion 133 provided in the second housing 130. The deodorizing lamp 160 may be disposed in front of the deodorizing filter 150. That is, the deodorizing lamp 160 may be disposed on the opposite side of the sterilizing lamp 140 with respect to the deodorizing filter 150. Therefore, the inlet 111, the sterilizing lamp 140, the deodorizing filter 150, and the deodorizing lamp 160 may be sequentially arranged along the flow direction of the air that is discharged from the duct outlet 61 of the cold air duct 60 and flows into the sterilization and deodorization apparatus 100.

The deodorizing lamp 160 may selectively activate the deodorizing filter 150. When it is required to sterilize and deodorize air inside the refrigerating compartment 22 by detecting a degree of contamination of the air inside the refrigerating compartment 22 or by detecting the opening and closing of the refrigerator compartment door 31, the sterilizing lamp 140 may be turned on for sterilization and deodorization operation. In response to the sterilizing lamp 140 being turned on and in response to ultraviolet rays being emitted from the sterilizing lamp 140, the air flowing into the inlet 111 may be sterilized. In addition, as the deodorizing filter 150 is activated by ultraviolet rays emitted from the sterilizing lamp 140, the air flowing into the inlet 111 may be deodorized. The air sterilized and deodorized by the sterilizing lamp 140 and the deodorizing filter 150 may be discharged through the outlet 170 (refer to FIGS. 1 and 2).

In response to determining that a sterilization required time elapses by determining the degree of contamination of the inside of the refrigerating compartment 22 and by determining an accumulated operation time of the sterilization/deodorization operation, the sterilizing lamp 140 may be turned off and the deodorizing lamp 160 may be turned on. That is, by turning off the sterilizing lamp 140 to stop sterilizing the air and by turning on the deodorizing lamp 160 to activate the deodorizing filter 150, the deodorization of air by the deodorizing filter 150 may be maintained.

In response to determining that the sterilization required time elapses by determining the degree of contamination of the inside of the refrigerating compartment 22 and the accumulated operation time of the sterilization/deodorization operation, the deodorizing lamp 160 may be turned off.

The sterilization and deodorization apparatus 100 may include the outlet 170. The outlet 170 may discharge air, which flows into the inlet 110 and then is sterilized and deodorized by the sterilizing lamp 140 and/or the deodorizing filter 150, into the refrigerating compartment 22 (refer to FIG. 2). The outlet 170 may be disposed in the second housing 130. The outlet 170 may be disposed between the deodorizing filter 150 and the deodorizing lamp 160. Therefore, the inlet 111, the sterilizing lamp 140, the deodorizing filter 150, the outlet 170, and the deodorizing lamp 160 may be sequentially arranged along the flow direction of the air that is discharged from the duct outlet 61 of the cold air duct 60 and flows into the sterilization and deodorization apparatus 100. As described above, because the inlet 111, the sterilizing lamp 140, the deodorizing filter 150, the outlet 170, and the deodorizing lamp 160 implement a straight flow path, it is possible to minimize and/or reduce loss of air flow during the sterilization and/or deodorization of air.

The outlet 170 may include a first outlet 171 formed in the shape of a hole between the deodorizing filter 150 and the deodorizing lamp 160. The first outlet 171 may be formed on the lower wall of the second housing 130. The air that flows into the inlet 111 and is sterilized and deodorized by the sterilizing lamp 140 and/or the deodorizing filter 150 may be discharged into the refrigerating compartment 22 (refer to FIG. 2) through the first outlet 171.

The outlet 170 may include a second outlet 173 formed on the left and right sides of the first outlet 171 by the deodorizing filter rib 134 and the deodorizing lamp rib 137. The air that flows into the inlet 111 and is sterilized and deodorized by the sterilizing lamp 140 and/or the deodorizing filter 150 may be guided by the deodorizing filter rib 134 and the deodorizing lamp rib 137 and discharged into the refrigerating compartment 22 (refer to FIG. 2) through the second outlet 173.

The sterilization and deodorization apparatus 100 may include an insulating tape 180 attached to the wall of the housing 110. The insulating tape 180 may be attached to the wall of the housing 110 adjacent to the refrigerating compartment 22 (refer to FIG. 2). The insulating tape 180 may be attached to the lower wall inside the second housing 130 adjacent to the refrigerating compartment 22 (refer to FIG. 2). The insulating tape 180 may dissipate heat generated from the sterilizing lamp 140.

The insulating tape 180 may include a reflective layer 181 provided to reflect ultraviolet rays emitted from the sterilizing lamp 140. The reflective layer 181 may be formed of a metal material. The reflective layer 181 may reflect ultraviolet rays emitted from the sterilizing lamp 140 and prevent and/or reduce the ultraviolet rays emitted from the sterilizing lamp 140 from directly reaching the surface of the housing 110. Particularly, when the housing 110 is formed of an injection molded product, the reflective layer 181 may prevent and/or reduce ultraviolet rays emitted from the sterilizing lamp 140 from directly reaching the surface of the housing 110, thereby preventing/reducing photodeterioration of the housing 110. In addition, the ultraviolet rays emitted from the sterilizing lamp 140 may be reflected by the reflective layer 181, thereby producing a re-sterilization effect of the air.

The insulating tape 180 may include an insulating layer 183 provided to prevent and/or reduce heat generated by the sterilizing lamp 140 from moving into the refrigerating compartment 22. The insulating layer 183 may prevent and/or reduce the heat, which is generated when the sterilizing lamp 140 emits ultraviolet rays, from moving into the refrigerating compartment 22 so as to prevent and/or reduce a temperature inside the refrigerating compartment 22 from rising.

As described above, as the insulating tape 180 is attached to the lower wall inside the second housing 130 adjacent to the refrigerating compartment 22 (refer to FIG. 2), the insulating tape 180 may prevent and/or reduce dew from forming on the surface of the second housing 130 adjacent to the refrigerating compartment 22 (refer to FIG. 2). Further, as the insulating tape 180 is attached to the lower wall inside the second housing 130 adjacent to the refrigerating compartment 22 (refer to FIG. 2), loss of output of the sterilizing lamp 140 may be prevented/reduced. Additionally, a lifespan of the sterilizing lamp 140 may be increased by attaching the insulating tape 180 to the lower wall inside the second housing 130 adjacent to the refrigerating compartment 22 (refer to FIG. 2).

The insulating tape 180 may be provided to allow the reflective layer 181 to be located above the insulating layer 183.

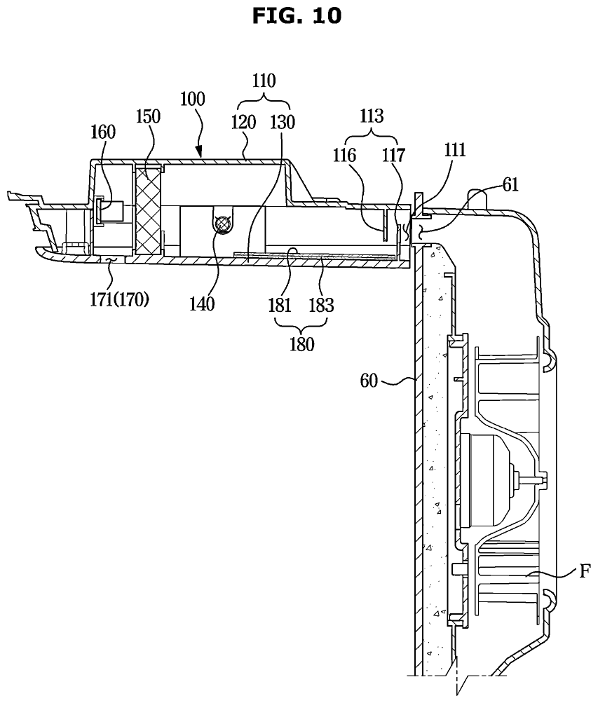

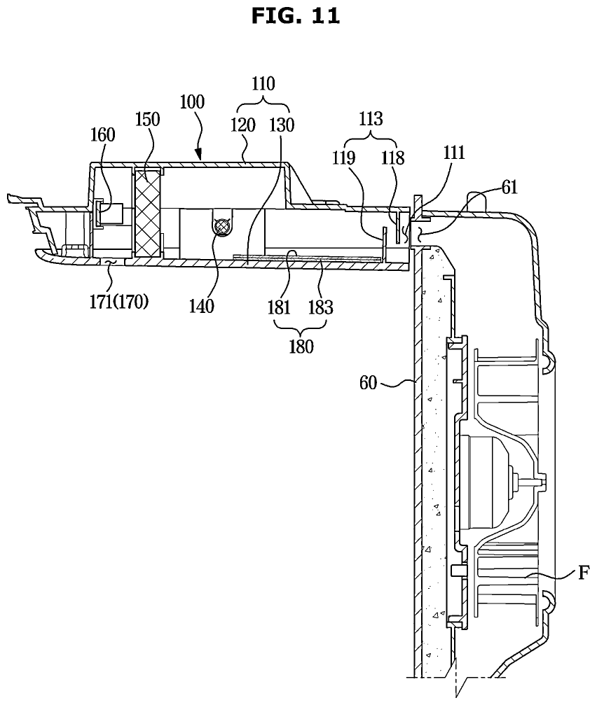

FIG. 10 is a cross-sectional view illustrating a state in which a first blocking rib provided in a portion adjacent to the inlet of the sterilization and deodorization apparatus is formed in front of a second blocking rib according to various embodiments. FIG. 11 is a cross-sectional view illustrating a state in which a second blocking rib provided in a portion adjacent to the inlet of the sterilization and deodorization apparatus is formed in front of a first blocking rib according to various embodiments.

As illustrated in FIG. 10, the housing 110 may include the blocking rib 113 provided to prevent and/or reduce ultraviolet rays emitted from the sterilizing lamp 140 from leaking out of the housing 110 through the inlet 111. The air flowing into the housing 110 may be sterilized by the ultraviolet rays emitted from the sterilizing lamp 140 mounted inside the housing 110. The ultraviolet rays emitted from the sterilizing lamp 140 may be prevented/reduced from leaking into the cold air duct 60 through the inlet 111 by the blocking rib 113.

The blocking rib 113 may be provided in the first housing 120 and the second housing 130 adjacent to the inlet 111, respectively. The blocking rib 113 may include a first blocking rib 116 formed in the first housing 120. The first blocking rib 116 may be formed to extend downward from the upper wall of the first housing 120.

The blocking rib 113 may include a second blocking rib 117 formed in the second housing 130. The second blocking rib 117 may be formed to extend upward from the lower wall of the second housing 130.

The first blocking rib 116 and the second blocking rib 117 may be formed at different positions with respect to the flow direction of air flowing into the inlet 111. The first blocking rib 116 may be formed in front of the second blocking rib 117. That is, the second blocking rib 117 may be formed closer to the inlet 111 than the first blocking rib 116. Because the first blocking rib 116 and the second blocking rib 117 are formed at different positions with respect to the flow direction of the air flowing into the inlet 111, the sum of a height of the first blocking rib 116 and a height of the second blocking rib 117 may be greater than a height of the inlet 111. Therefore, the ultraviolet rays emitted from the sterilizing lamp 140 may be completely blocked from leaking into the cold air duct 60 through the inlet 111 by the first blocking rib 116 and the second blocking rib 117.

As illustrated in FIG. 11, the housing 110 may include the blocking rib 113 provided to prevent and/or reduce ultraviolet rays emitted from the sterilizing lamp 140 from leaking out of the housing 110 through the inlet 111. The air flowing into the housing 110 may be sterilized by ultraviolet rays emitted from the sterilizing lamp 140 mounted inside the housing 110. The ultraviolet rays emitted from the sterilizing lamp 140 may be prevented/reduced from leaking into the cold air duct 60 through the inlet 111 by the blocking rib 113.

The blocking rib 113 may be provided in the first housing 120 and the second housing 130 adjacent to the inlet 111, respectively. The blocking rib 113 may include a first blocking rib 118 formed in the first housing 120. The first blocking rib 118 may be formed to extend downward from the upper wall of the first housing 120.

The blocking rib 113 may include a second blocking rib 119 formed in the second housing 130. The second blocking rib 119 may be formed to extend upward from the lower wall of the second housing 130.

The first blocking rib 118 and the second blocking rib 119 may be formed at different positions with respect to the flow direction of air flowing into the inlet 111. The second blocking rib 119 may be formed in front of the first blocking rib 118. That is, the first blocking rib 118 may be formed closer to the inlet 111 than the second blocking rib 119. Because the first blocking rib 118 and the second blocking rib 119 are formed at different positions with respect to the flow direction of the air flowing into the inlet 111, the sum of a height of the first blocking rib 118 and a height of the second blocking rib 119 may be greater than a height of the inlet 111. Therefore, the ultraviolet rays emitted from the sterilizing lamp 140 may be completely blocked from leaking into the cold air duct 60 through the inlet 111 by the first blocking rib 118 and the second blocking rib 119.

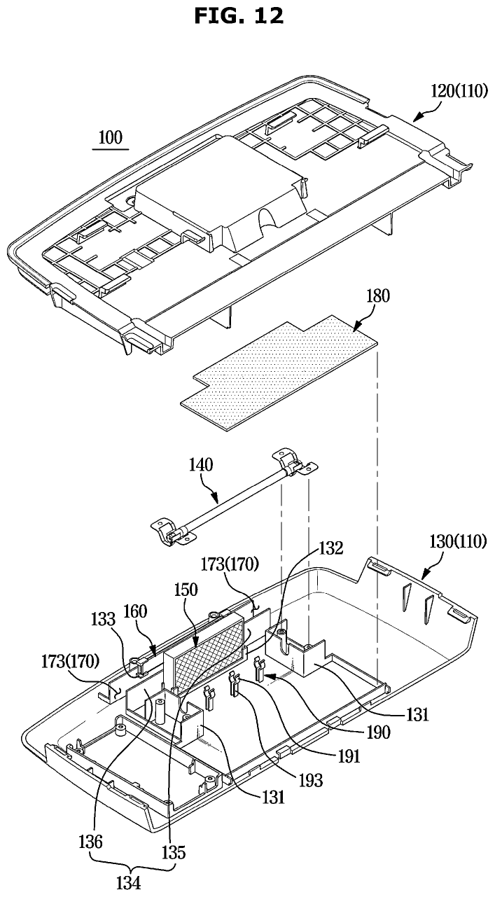

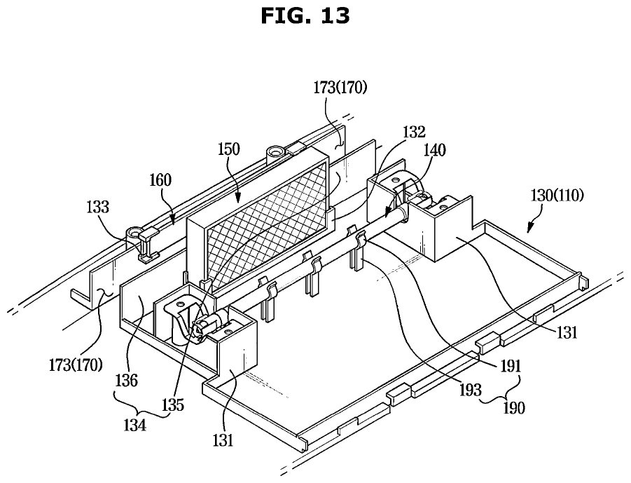

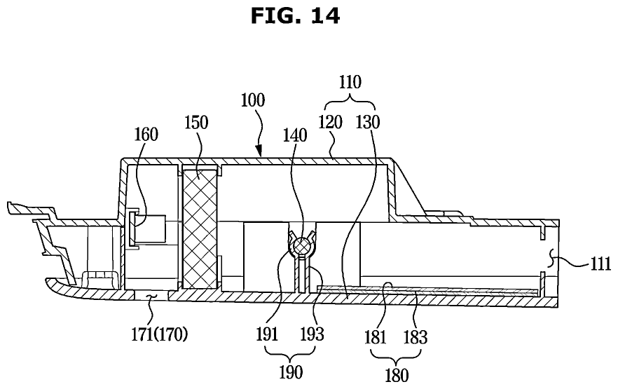

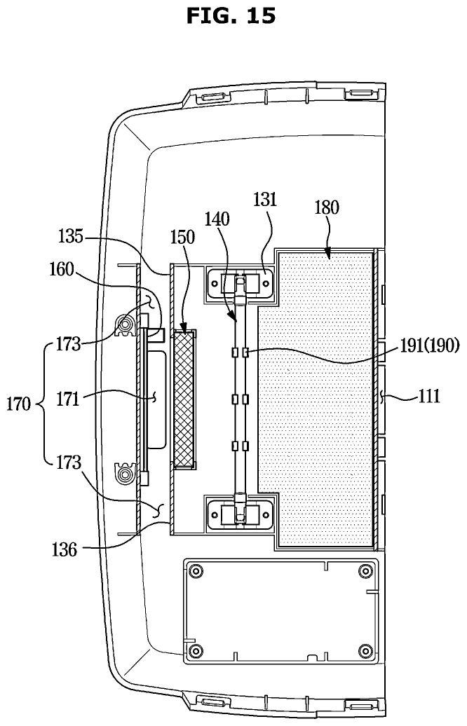

FIG. 12 is an exploded perspective view of the sterilization and deodorization apparatus including a heat dissipation support supporting the sterilizing lamp according to various embodiments. FIG. 13 is a perspective view illustrating a state in which the heat dissipation support supports the sterilizing lamp according to various embodiments. FIG. 14 is a cross-sectional view of the sterilization and deodorization apparatus including the heat dissipation support supporting the sterilizing lamp according to various embodiments. FIG. 15 is a diagram illustrating a top plan view of the inside of the second housing in which the sterilizing lamp, the deodorizing filter, and the deodorizing lamp are installed, the heat dissipation support supports the sterilizing lamp, and the insulating tape is attached, according to various embodiments.

As illustrated in FIGS. 12, 13, 14 and 15 (which may be referred to as FIGS. 12 to 15), the sterilization and deodorization apparatus 100 may include a heat dissipation support 190 provided to support the sterilizing lamp 140 formed in a cylindrical shape. The heat dissipation support 190 may be formed of a material capable of transmitting heat generated by the sterilizing lamp 140. The heat dissipation support 190 may be formed of a metal material. A plurality of heat dissipation supports 190 may be provided. The heat dissipation support 190 may be provided to be in contact with the sterilizing lamp 140 so as to dissipate heat generated from the sterilizing lamp 140.

The heat dissipation support 190 may include a contact portion 191 provided to be in contact with the sterilizing lamp 140. The contact portion 191 may be provided to surround a portion of the sterilizing lamp 140.

The heat dissipation support 190 may include a support portion 193 connected to the contact portion 191. One end of the support portion 193 may be connected to the contact portion 191 to support the sterilizing lamp 140, and the other end thereof may be fixed to the wall of the housing 110 adjacent to the refrigerating compartment 22. At least a portion of the support portion 193 may be provided to be in contact with the wall of the housing 110 adjacent to the refrigerating compartment 22. The housing 110 adjacent to the refrigerating compartment 22 may be the second housing 130. Accordingly, at least a portion of the support portion 193 may be provided to be in contact with the lower wall of the second housing 130 (refer to FIG. 2).

As described above, as the heat dissipation support 190 formed of metal is fixed to the lower wall inside the second housing 130 adjacent to the refrigerating compartment 22, it is possible to prevent and/or reduce dew from forming on the surface of the second housing 130 adjacent to the refrigerating compartment 22. Further, as the heat dissipation support 190 formed of metal is fixed to the lower wall inside the second housing 130 adjacent to the refrigerating compartment 22, loss of output of the sterilizing lamp 140 may be prevented/reduced. Further, as the heat dissipation support 190 formed of metal is fixed to the lower wall inside the second housing 130 adjacent to the refrigerating compartment 22, a lifespan of the sterilizing lamp 140 may be increased (refer to FIG. 2).

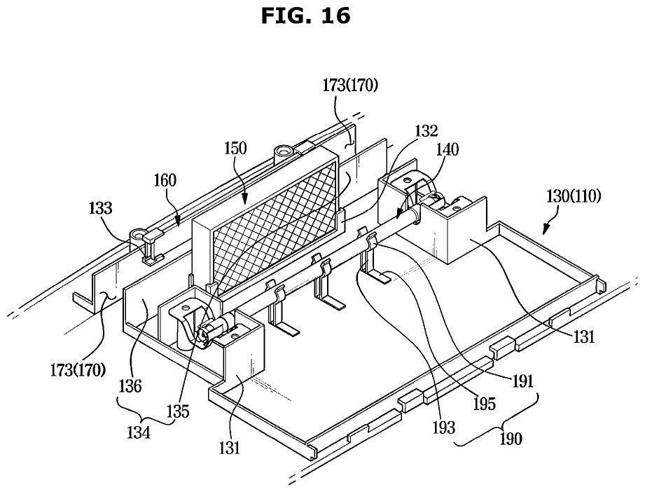

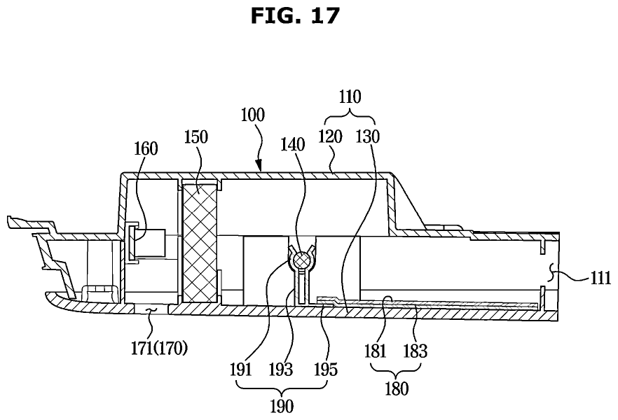

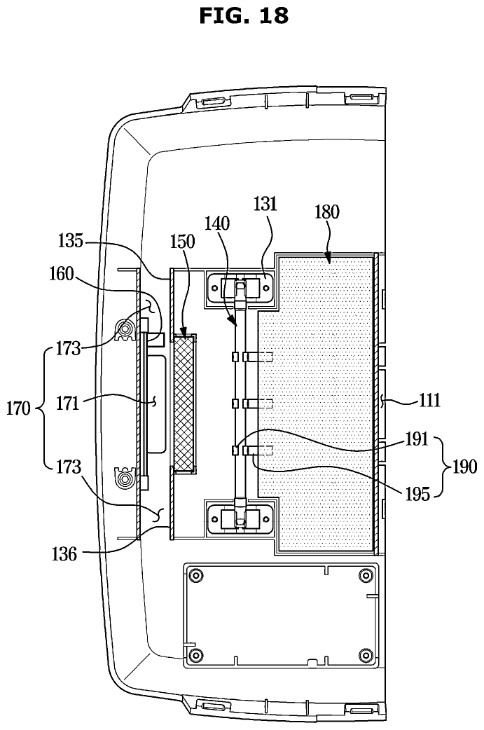

FIG. 16 is a perspective view illustrating a state in which the heat dissipation support supporting the sterilizing lamp includes an extension portion extending from a support portion according to various embodiments. FIG. 17 is a cross-sectional view of the sterilization and deodorization apparatus including the heat dissipation support including the extension portion according to various embodiments. FIG. 18 is a diagram illustrating a top plan view of the inside of the second housing in which the sterilizing lamp, the deodorizing filter, and the deodorizing lamp are installed, the heat dissipation support including the extension portion supports the sterilizing lamp, and the insulating tape is attached, according to various embodiments.

As illustrated in FIGS. 16, 17 and 18 (which may be referred to as FIGS. 16 to 18), the heat dissipation support 190 may include an extension portion 195 extending from the support portion 193 along the wall of the housing 110. The extension portion 195 may extend along the surface of the lower wall of the second housing 130. The extension portion 195 may extend in a direction parallel to the flow direction of air flowing into the sterilization and deodorization apparatus 100. That is, the extension portion 195 may extend in a direction parallel to the flow direction of air that flows into the sterilization and deodorization apparatus 100 through the inlet 111. In the drawing, it is illustrated that a length from the extension portion 195 to the support portion 193 is similar to a length of the support portion 193, but is not limited thereto. That is, the extension portion 195 may be provided to extend from the support portion 193 to the inlet 111. The extension portion 195 may be provided to have the same width as the support portion 193.

The insulating tape 180 may be attached to the wall of the housing 110 to cover an upper portion of the extension portion 195. The insulating tape 180 may be attached to the wall of the housing 110 to cover a portion of the upper portion of the extension portion 195. That is, the insulating tape 180 attached to the lower wall inside the second housing 130 may be attached to the lower wall inside the second housing 130 to cover a portion of the upper portion of the extension portion 195. In the drawing, it is illustrated that the insulating tape 180 is attached to the lower wall inside the second housing 130 to cover a portion of the upper portion of the extension portion 195, but is not limited thereto. That is, the insulating tape 180 may be attached to the lower wall inside the second housing 130 to cover the entire upper portion of the extension portion 195.

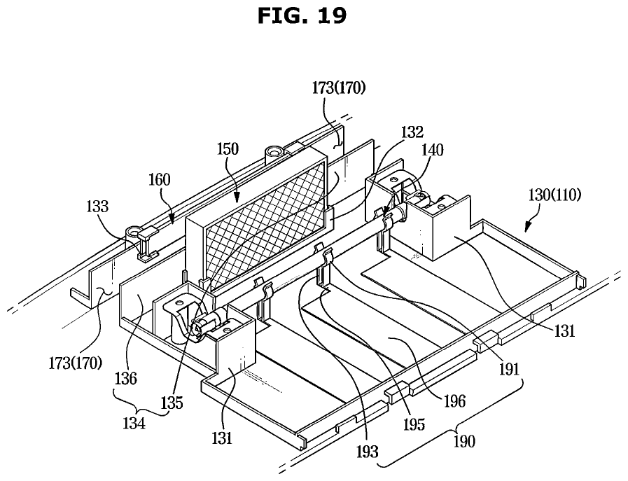

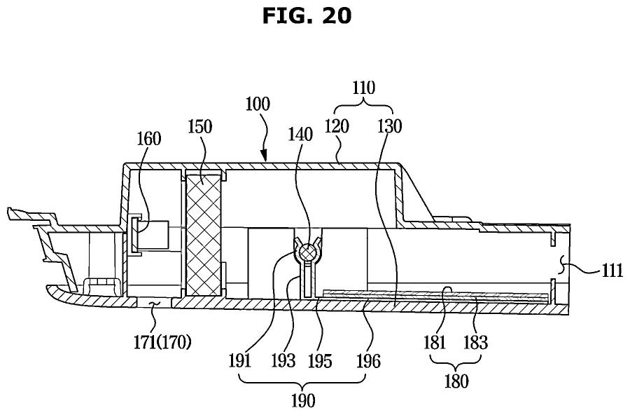

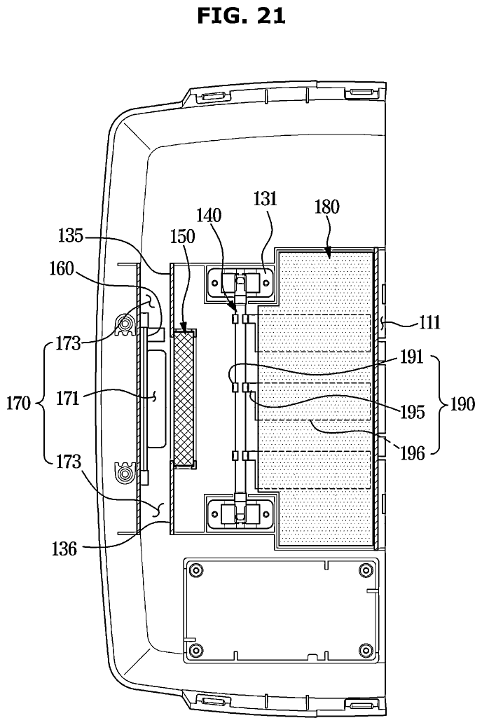

FIG. 19 is a perspective view illustrating a state in which the extension portion of the heat dissipation support includes an expansion portion having a width greater than a width of the support portion according to various embodiments. FIG. 20 is a cross-sectional view of the sterilization and deodorization apparatus including the heat dissipation support including the expansion portion according to various embodiments. FIG. 21 is a diagram illustrating a top plan view of the inside of the second housing in which the sterilizing lamp, the deodorizing filter, and the deodorizing lamp are installed, the heat dissipation support including the expansion portion supports the sterilizing lamp, and the insulating tape is attached, according to various embodiments.

As illustrated in FIGS. 19, 20 and 21 (which may be referred to as FIGS. 19 to 21), the extension portion 195 of the heat dissipation support 190 may include an expansion portion 196 provided to have a width that is greater than the width of the support portion 193. In the same manner as the extension portion 195, the expansion portion 196 may extend along the surface of the lower wall of the second housing 130. The expansion portion 196 may extend in a direction parallel to the flow direction of air flowing into the sterilization and deodorization apparatus 100. That is, the expansion portion 196 may extend in a direction parallel to the flow direction of air that flows into the sterilization and deodorization apparatus 100 through the inlet 111. In the drawings, it is illustrated that the expansion portion 196 extends to a position adjacent to the inlet 111, but is not limited thereto.

The insulating tape 180 may be attached to the wall of the housing 110 to cover an upper portion of the expansion portion 196. The insulating tape 180 may be attached to the wall of the housing 110 to cover a portion of the upper portion of the expansion portion 196. That is, the insulating tape 180 attached to the lower wall inside the second housing 130 may be attached to the lower wall inside the second housing 130 to cover a portion of the upper portion of the expansion portion 196. In the drawing, it is illustrated that the insulating tape 180 is attached to the lower wall inside the second housing 130 to cover a portion of the upper portion of the expansion portion 196, but is not limited thereto. That is, the insulating tape 180 may be attached to the lower wall inside the second housing 130 so as to cover the entire upper portion of the expansion portion 196.

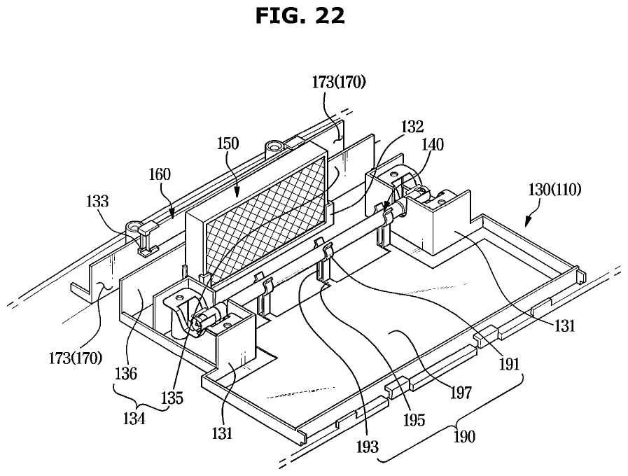

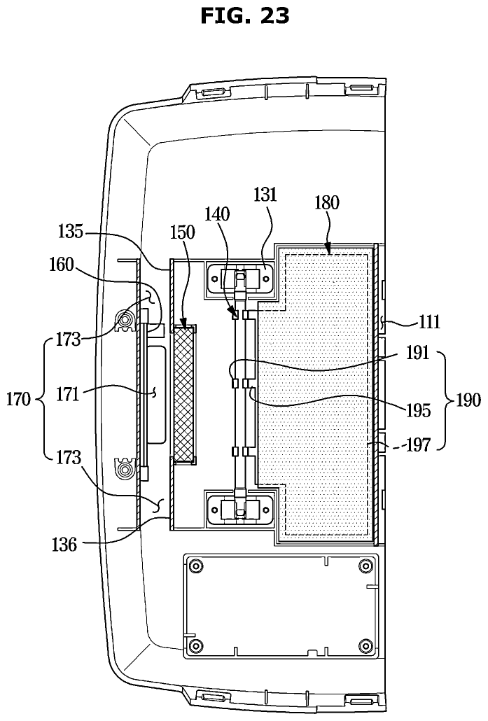

FIG. 22 is a perspective view illustrating a state in which expansion portions of the heat dissipation support are connected to each other in a direction perpendicular to a flow direction of air according to various embodiments. FIG. 23 is a diagram illustrating a top plan view of the inside of the second housing in which the sterilizing lamp, the deodorizing filter, and the deodorizing lamp are installed, the heat dissipation support including the expansion portions, which are connected to each other in the direction perpendicular to the flow direction of air, supports the sterilizing lamp, and the insulating tape is attached, according to various embodiments.

As illustrated in FIGS. 22 and 23, the extension portion 195 of the heat dissipation support 190 may include an expansion portion 197 provided to have a width that is greater than the width of the support portion 193. The expansion portions 197 may be provided to be connected to each other in a direction perpendicular to the flow direction of air flowing into the sterilization and deodorization apparatus 100. That is, the expansion portions 197 may be provided to be connected to each other in a direction perpendicular to the flow direction of air that flows into the sterilization and deodorization apparatus 100 through the inlet 111. The expansion portion 197 may extend along the surface of the lower wall of the second housing 130 in the same manner as the extension portion 195. The expansion portion 197 may extend in a direction parallel to the flow direction of air flowing into the sterilization and deodorization apparatus 100. That is, the expansion portion 197 may extend in a direction parallel to the flow direction of air that flows into the sterilization and deodorization apparatus 100 through the inlet 111. In the drawing, it is illustrated that the extension 197 extends to a position adjacent to the inlet 111, but is not limited thereto.

According to an example embodiment, the refrigerator may include: a cabinet, a storage compartment disposed inside the cabinet, a cold air duct disposed behind the storage compartment, and a sterilization and deodorization apparatus mounted inside the storage compartment, and configured to draw air, discharged from the cold air duct, sterilize and deodorize the air, and discharge the sterilized and deodorized air into the storage compartment. The sterilization and deodorization apparatus may include: an inlet connected to a duct outlet of the cold air duct, a deodorizing filter configured to deodorize the air flowing into the inlet, the sterilizing lamp disposed between the inlet and the deodorizing filter and configured to sterilize the air flowing into the inlet, an outlet configured to discharge air sterilized and deodorized by the sterilizing lamp and the deodorizing filter into the storage compartment, and a deodorizing lamp disposed opposite to the sterilizing lamp with respect to the deodorizing filter and configured to activate the deodorizing filter.

The inlet, the sterilizing lamp, the deodorizing filter, and the outlet may be sequentially arranged along a flow direction of air discharged from the duct outlet and flowing into the sterilization and deodorization apparatus.

The outlet may be disposed between the deodorizing filter and the deodorizing lamp.

The sterilizing lamp may have a cylindrical shape.

A sterilizing area in which the air flowing into the inlet is sterilized by the sterilizing lamp, may include the first sterilizing area formed in a section between the inlet and the sterilizing lamp, and a second sterilizing S2 formed in a section between the sterilizing lamp and the deodorizing filter.

A distance between the inlet and the sterilizing lamp that is the first sterilizing area may be greater than the distance between the sterilizing lamp and the deodorizing filter that is the second sterilizing area.

The sterilization and deodorization apparatus may further include a housing mounted on an upper wall inside the storage compartment.

The housing may include a first housing mounted on the upper wall inside the storage compartment, and a second housing mounted on the lower portion of the first housing, and forming the outlet, the second housing on which the sterilizing lamp, the deodorizing filter, and the deodorizing lamp are mounted.

An inlet may be formed by the first housing and the second housing. A blocking rib configured to prevent and/or reduce ultraviolet rays emitted from the sterilizing lamp from leaking out through the inlet may be provided in the first housing and the second housing adjacent to the inlet.

The blocking rib may include a first blocking rib 114 formed to extend downward from the upper wall of the first housing, and a second blocking rib extending upward from the lower wall of the second housing.

The first blocking rib and the second blocking rib may be disposed at a same position with respect to the flow direction of air flowing into the inlet.

The first blocking ribs and the second blocking ribs may be disposed at different positions with respect to the flow direction of air flowing into the inlet.

The second housing may include a sterilizing lamp mounting portion on which the sterilizing lamp is configured to be mounted, a deodorizing filter mounting portion on which the deodorizing filter is configured to be mounted, a deodorizing lamp mounting portion on which the deodorizing lamp is configured to be mounted, the deodorizing filter rib disposed on left and right sides of the deodorizing filter, respectively, and configured to prevent and/or reduce ultraviolet rays emitted from the sterilizing lamp from leaking through the outlet, and the deodorizing lamp rib disposed in front of the deodorizing lamp and configured to prevent and/or reduce ultraviolet rays emitted from the deodorizing lamp from leaking through the outlet.

The deodorizing filter rib may include a first deodorizing filter rib disposed on the left side of the deodorizing filter, and a second deodorizing filter rib disposed on the right side of the deodorizing filter. A length between the left end of the first deodorizing filter rib and the right end of the second deodorizing filter rib may be greater than a length of the sterilizing lamp.

A length of the deodorizing lamp rib may be greater than a length of the deodorizing filter.

According to an example embodiment, a sterilization and deodorization apparatus configured to draw air, discharged from a duct outlet of a cold air duct disposed behind a storage compartment, and sterilize and deodorize the air, and discharge the sterilized and deodorized air into the storage compartment, may include: a housing mounted inside the storage compartment, an inlet connected to the duct outlet, a deodorizing filter configured to deodorize air flowing into the inlet, a sterilizing lamp having a cylindrical shape and disposed between the inlet and the deodorizing filter and configured to sterilize air flowing into the inlet, an outlet configured to discharge air sterilized and deodorized by the sterilizing lamp and the deodorizing filter into the storage compartment, and a deodorizing lamp disposed opposite to the sterilizing lamp with respect to the deodorizing filter and configured to activate the deodorizing filter.

The housing may include a first housing mounted on an upper wall inside the storage compartment, and a second housing mounted on a lower portion of the first housing, and provided to form the outlet, the second housing on which the sterilizing lamp, the deodorizing filter, and the deodorizing lamp are mounted.

The second housing may include a deodorizing filter rib disposed on left and right sides of the deodorizing filter, respectively, and configured to prevent and/or reduce ultraviolet rays emitted from the sterilizing lamp from leaking through the outlet, and a deodorizing lamp rib disposed in front of the deodorizing lamp and configured to prevent and/or reduce ultraviolet rays emitted from the deodorizing lamp from leaking through the outlet.

The outlet may include a first outlet having a shape of a hole between the deodorizing filter and the deodorizing lamp, and a second outlet formed on the left and right sides of the first outlet by the deodorizing filter rib and the deodorizing lamp rib.

The inlet, the sterilizing lamp, the deodorizing filter, the outlet and the deodorizing lamp may be sequentially arranged along the flow direction of air discharged from the duct outlet and flowing into the sterilization and deodorization apparatus.

As is apparent from the above description, it is possible to sufficiently secure a sterilization area and a sterilization time of air flowing into an inlet.

Further, it is possible to simultaneously perform sterilization and/or deodorization of air using a sterilizing lamp.

Further, it is possible to improve power consumption.

Further, it is possible to minimize and/or reduce loss of air flow during sterilization and/or deodorization of air that flows into an inlet.

Further, it is possible to minimize and/or reduce a volume of a sterilization and deodorization apparatus.

Further, it is possible to prevent and/or reduce ultraviolet rays, which are emitted from a sterilizing lamp, from leaking out of a housing through an inlet and an outlet.

While the present disclosure has been illustrated described with reference to various example embodiments, it will be understood that the various example embodiments are intended to be illustrative, not limiting. It will be further understood by those skilled in the art that various changes in form and detail may be made without departing from the true spirit and full scope of the present disclosure, including the appended claims and their equivalents. It will also be understood that any of the embodiment(s) described herein may be used in conjunction with any other embodiment(s) described herein.

What is claimed is:

1. A refrigerator comprising:
a cabinet;
a storage compartment disposed inside the cabinet;
a cold air duct disposed behind the storage compartment; and
a sterilization and deodorization apparatus mounted inside the storage compartment, and configured to draw air discharged from the cold air duct, sterilize and deodorize the air, and discharge the sterilized and deodorized air into the storage compartment,
wherein the sterilization and deodorization apparatus comprises:
an inlet connected to a duct outlet of the cold air duct;
a deodorizing filter configured to deodorize the air flowing into the inlet;
a sterilizing lamp disposed between the inlet and the deodorizing filter and configured to sterilize the air flowing into the inlet;
an outlet configured to discharge air sterilized and deodorized by the sterilizing lamp and the deodorizing filter into the storage compartment; and
a deodorizing lamp disposed opposite to the sterilizing lamp with respect to the deodorizing filter and configured to activate the deodorizing filter.

2. The refrigerator of claim 1, wherein
the inlet, the sterilizing lamp, the deodorizing filter, and the outlet are sequentially arranged along a flow direction of air discharged from the duct outlet and flowing into the sterilization and deodorization apparatus.

3. The refrigerator of claim 1, wherein
the outlet is disposed between the deodorizing filter and the deodorizing lamp.

4. The refrigerator of claim 1, wherein
the sterilizing lamp has a cylindrical shape.

5. The refrigerator of claim 1, wherein
a sterilizing area in which the air flowing into the inlet is configured to be sterilized by the sterilizing lamp, comprises a first sterilizing area in a section between the inlet and the sterilizing lamp and a second sterilizing area in a section between the sterilizing lamp and the deodorizing filter.

6. The refrigerator of claim 5, wherein
a distance between the inlet and the sterilizing lamp that is the first sterilizing area is greater than a distance between the sterilizing lamp and the deodorizing filter that is the second sterilizing area.

7. The refrigerator of claim 1, wherein
the sterilization and deodorization apparatus further comprises a housing mounted on an upper wall inside the storage compartment.

8. The refrigerator of claim 7, wherein
the housing comprises:
a first housing mounted on the upper wall inside the storage compartment; and
a second housing mounted on a lower portion of the first housing, and providing the outlet, and the second housing on which the sterilizing lamp, the deodorizing filter, and the deodorizing lamp are mounted.

9. The refrigerator of claim 8, wherein
the inlet is formed by the first housing and the second housing,
wherein a blocking rib configured to prevent and/or reduce ultraviolet rays emitted from the sterilizing lamp from leaking out through the inlet is provided in the first housing and the second housing adjacent to the inlet.

10. The refrigerator of claim 9, wherein
the blocking rib comprises:
a first blocking rib extending downward from an upper wall of the first housing; and
a second blocking rib extending upward from a lower wall of the second housing.

11. The refrigerator of claim 10, wherein
the first blocking rib and the second blocking rib are disposed at the same position with respect to a flow direction of air flowing into the inlet.

12. The refrigerator of claim 10, wherein
the first blocking rib and the second blocking rib are disposed at different positions with respect to a flow direction of air flowing into the inlet.

13. The refrigerator of claim 8, wherein
the second housing comprises:
a sterilizing lamp mounting portion on which the sterilizing lamp is configured to be mounted;
a deodorizing filter mounting portion on which the deodorizing filter is configured to be mounted;
a deodorizing lamp mounting portion on which the deodorizing lamp is configured to be mounted;
a deodorizing filter rib disposed on left and right sides of the deodorizing filter, respectively, and configured to prevent and/or reduce ultraviolet rays emitted from the sterilizing lamp from leaking through the outlet; and
a deodorizing lamp rib disposed in front of the deodorizing lamp and configured to prevent and/or reduce ultraviolet rays emitted from the deodorizing lamp from leaking through the outlet.

14. The refrigerator of claim 13, wherein
the deodorizing filter rib comprises:
a first deodorizing filter rib disposed on the left side of the deodorizing filter; and
a second deodorizing filter rib disposed on the right side of the deodorizing filter,
wherein a length between a left end of the first deodorizing filter rib and a right end of the second deodorizing filter rib is greater than a length of the sterilizing lamp.

15. The refrigerator of claim 13, wherein
a length of the deodorizing lamp rib is greater than a length of the deodorizing filter.
